(19) United States
(12) Patent Application Publication
(10) Pub. No.: US 2008/0033411 A1
(43) Pub. Date: Feb. 7, 2008

(54) High efficiency electromagnetic laser energy cutting device

(75) Inventors: Andriasyan Manvel Artyom, San Diego, CA (US); Dmitri Boutoussov, Dana Point, CA (US)

Correspondence Address: Kenton R. Mullins;Stout, Uxa, Buyan & Mullins, LLP, Suite 300, 4 Venture, Irvine, CA, 92618, US

(73) Assignee: BioLase Technology, Inc., Irvine, CA (US)

(21) Appl. No.: 11/487,112

(22) Filed: Jul. 14, 2006

(51) Int. Cl. A61B 18/18

(52) U.S. Cl. 606/10

(57) ABSTRACT

A medical laser is described that contains a modulator or saturable absorber. The laser produces output optical energy suitable for cutting tissue while minimizing wasted output optical energy that could result in unnecessary pain to a patient. The medical laser described enables efficient, effective cutting of tissue.

### BACKGROUND OF THE INVENTION

1. Field of the Invention

The present invention relates generally to electromagnetic procedural devices and, more particularly, to medical laser devices adapted for ablating tissue.

2. Description of Related Art

A variety of electromagnetic laser energy generating architectures have existed in the prior art. Laser devices based upon these architectures have found application in the practice of, for example, dentistry, wherein laser cutting devices have resulted in more precise cutting with greater patient comfort than had been possible with, for example, high-speed dental drills. Elements of a typical solid-state laser device are shown in FIG. 1. The illustrated device generally comprises a laser rod 10 for emitting coherent light, the laser rod 10 having an optical axis 15. A source such as a diode or a flashlamp (not shown) typically stimulates the laser rod 10 to emit the coherent light. The illustrated laser device further comprises a high-reflectivity (HR) mirror 20 and an output coupling (OC) element 25 oriented to generate output optical energy 30.

The output optical energy produced by the prior art laser device shown in FIG. 1 may take a form shown in FIG. 2. This illustrated output comprises a relatively large initial pulse 50 followed by gradually rising energy in a form of spikes or micropulses distributed essentially continuously in time. The gradually rising energy ramps up to a maximum energy and subsequently decreases over time.

In order for cutting of a given tissue to occur, the energy, represented as Relative Pulse Amplitude in FIG. 2, must exceed a level, which may be referred to as an ablation threshold 55 of the given tissue. A portion of the illustrated energy distribution, which may be referred to as an effective portion 60, exceeds the ablation threshold 55 and so may be effective in cutting the given tissue.

When employed in cutting applications as may occur, for example, in the practice of dentistry, however, the prior art energy distribution over time illustrated in FIG. 2 can suffer an important disadvantage. In particular, energy not exceeding the ablation threshold 55, e.g., a portion that may be referred to as tail energy 65 in FIG. 2, does not substantially contribute to cutting. The tail energy 65, rather, may serve to dehydrate a surface of a target (e.g., a tooth) so that when a subsequent energy pulse (similar to that shown in FIG. 2) impacts the tooth, the surface of the tooth may be undesirably altered due in certain instances to an effect of the tail energy 65. That is, the target surface may, for example, be dehydrated, hardened, modified, or even melted or fused, to some degree, so that the subsequent energy pulse may in certain instances be less able to cut the tissue effectively without, for example, increased pain to a patient. Additionally, the tail energy 65 can be considered essentially wasted in some applications, thereby contributing to general inefficiency of operation of the laser device. Further, in certain cases part or all of the tail energy 65 is transferred in a form of heat to the tissue of the patient, thereby potentially producing increased pain to the patient.

A need thus exists in the prior art to reduce sub-ablation-threshold energy emitted by electromagnetic energy procedural devices. A further need exists for a medical device capable of efficiently cutting tissue while emitting reduced sub-ablation-threshold energy.

### SUMMARY OF THE INVENTION

The present invention addresses these needs by providing a medical procedural device (e.g., laser) that can generates energy (e.g., optical energy, a greatest portion of which has a level greater than an ablation threshold. An embodiment of the invention herein disclosed may be implemented as an optical resonator comprising a laser rod having an optical axis, a high-reflectivity optical element disposed within a path of the optical axis near a first end of the laser rod, and an output coupling element disposed within the path of the optical axis nearer to a second end of the laser rod than to the first end of the laser rod. A modulator is disposed in the path of the optical axis, the modulator being adapted to influence energy pulses emitted by the optical resonator. Substantially all energy pulses generated by the optical resonator that have an amplitude not exceeding the ablation threshold have an amplitude significantly less than the ablation threshold. In this way, wasted output optical energy is reduced, resulting potentially in greater efficiency and greater patient comfort.

The present invention may take a form of a medical laser capable of generating optical energy, a significant portion, such as a majority, of which has a level greater than the ablation threshold. One embodiment of the medical laser may comprise a laser rod having an optical axis, a high-reflectivity optical element disposed within a path of the optical axis near a first end of the laser rod, and an output coupling element disposed within the path of the optical axis nearer to a second end of the laser rod than to the first end of the laser rod, wherein the embodiment further may comprise a saturable absorber disposed in the path of the optical axis.

Another embodiment of the present invention may be implemented as an erbium, e.g., an erbium, chromium, yttrium, scandium, gallium, garnet (Er, Cr:YSGG), medical laser adapted to cutting tissue, the laser including a laser rod comprising an erbium crystal and having an optical axis, the laser rod further having a first end and a second end. The embodiment can comprise a high-reflectivity optical element disposed within a path of the optical axis near the first end and an output coupling element disposed within the path of the optical axis nearer to the second end than to the first end. A modulator formed of, for example, crystalline iron-doped zinc selenide may be disposed within the path of the optical axis. This embodiment may generate output optical energy such that a greatest portion of the optical energy has a level greater than an ablation threshold.

While the apparatus has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a pictorial diagram of a prior-art laser that may be employed in medical cutting applications;

FIG. 2 is a plot illustrating a form of laser pulses generated by the prior-art laser of FIG. 1;

FIG. 3 illustrates a medical laser including a modulator element disposed at a Brewster angle according to an embodiment of the present invention;

FIG. 4 is a plot describing laser pulses generated by an embodiment of the present invention;

FIG. 5 depicts an alternative embodiment of the present invention wherein the modulator is disposed in a vertical orientation;

FIG. 6 illustrates another embodiment of the present invention wherein a high-reflectivity optical component of the laser comprises the modulator;

FIG. 7 describes yet another embodiment of the present invention having the modulator incorporated into an output coupling element;

FIG. 8 is a representation of an oscilloscope trace of laser pulses produced by an embodiment of the present invention;

FIG. 9 is a sketch of an oscilloscope trace showing a single pulse generated by an embodiment of the present invention; and

FIG. 10 is a block diagram showing a fluid output used in combination with an electromagnetic energy source having a driving circuit.

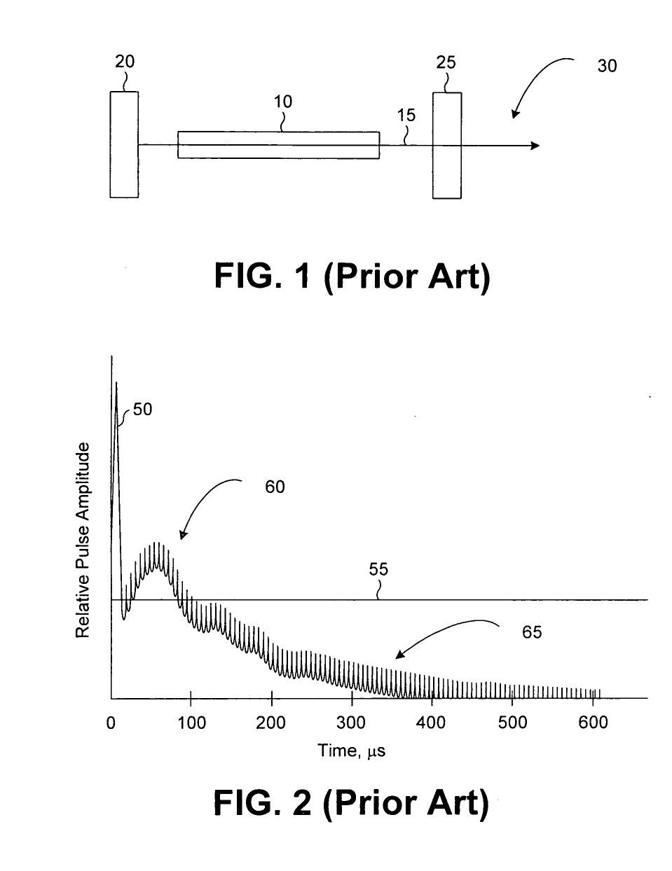

### DETAILED DESCRIPTION OF THE INVENTION

Reference will now be made in detail to the exemplary embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. The present invention may be practiced in conjunction with various techniques that are conventionally used in the art, and only so much of the common elements are included herein as are necessary to provide an understanding of the present invention. The present invention has applicability in the field of electromagnetic procedural devices in general. For illustrative purposes, however, the following description pertains to a medical laser device employed in dental applications.

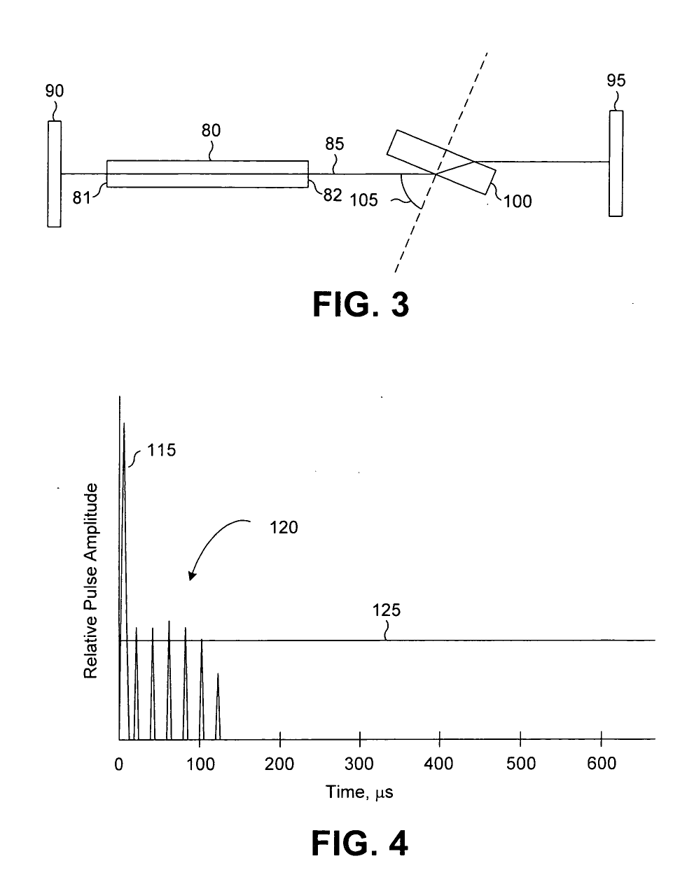

With further reference to the drawings, FIG. 3 illustrates an embodiment of the medical procedural device of the present invention, which may comprise a medical laser that produces output optical energy having a distribution over time that can be relatively efficient and that cam provide for improved patient comfort in tissue cutting applications. The illustrated embodiment, which may be referred to as an optical resonator, can comprise a laser rod 80 having an optical axis 85, a high-reflectivity (HR) optical element, e.g., a high-reflectivity mirror, 90, and an output coupling (OC) element 95, disposed generally in a path of the optical axis 85. As shown, the HR 90 is disposed near a first end 81 of the laser rod 80, and the OC 95 is disposed nearer to a second end 82 of the laser rod 80 than to the first end 81. According to a representative embodiment, the laser rod 80 comprises an erbium crystal, such as an erbium, chromium, yttrium, scandium, gallium, garnet (Er, Cr:YSGG) crystal operated at a wavelength of about 3 microns. The HR 90 and the OC 95 in the illustrated embodiment may be formed of zinc selenide material that is transparent to a wavelength of interest (e.g., 3 microns).

The embodiment further comprises a modulator 100, which may constitute a saturable absorber, disposed in the path of the optical axis 85. The modulator 100 may be formed of, for example, crystalline iron-doped zinc selenide, Fe:ZnSe. According to one embodiment, the modulator 100 is circular with a diameter of about 5 millimeters (mm) and a thickness of about 2 mm. According to another embodiment, the modulator 100 may be rectangular. These dimensions may be compared with dimensions of the HR 90 and OC 95, each of which may have a diameter of about 20 mm and a thickness of about 2 mm. The laser rod may be about 70 mm in length and may have a diameter of about 3 mm. The modulator 100 may be secured using any known crystal holder inside the optical resonator. According to another embodiment, the modulator 100 can have dimensions of about 8.02 mm×4.16 mm×1.13 mm, and initial transmission of the Fe:ZnSe can be about 59.8%. A representative embodiment employs an OC 95 having a reflectivity of about 86%.

As is more fully described below, the modulator 100 may be positioned anywhere along the optical axis 85. In the illustrated embodiment, the modulator 100 is disposed between the second end 82 of the laser rod 80 and the OC 95. Further the modulator 100 is oriented at a Brewster angle 105, which, for Fe:ZnSe, can be about 67.7 degrees.

Methods are known for the fabrication of the modulator 100, which can take a form of a single crystal of Fe:ZnSe or which can be a polycrystalline (i.e., formed of multiple small crystals fused together) form of Fe:ZnSe. For example, a fabrication procedure that may be employed in manufacture of the present invention is described in J. Kermal, V. V. Fedorov, A. Gallian, and S. B. Mirov, “3.9-4.8 μm Gain-Switched Lasing of Fe:ZnSe at Room Temperature,” Optics Express, 26 Dec. 2005, Vol. 13, No. 26, pp. 10608-10615.

The modulator 100 in the embodiment of the optical resonator may beneficially influence optical energy generated by the optical resonator. FIG. 4 is a chart of pulse amplitude representing output optical energy produced by the optical resonator over time. The chart in FIG. 4 illustrates an ablation threshold 125, which should be compared with the ablation threshold 55 presented in the chart of FIG. 2. The output optical energy illustrated in FIG. 4 includes a relatively large initial micropulse 115 (which may be referred to below as a high-intensity leading micropulse) followed by an effective portion 120, i.e., a portion of energy lying above the ablation threshold 125, comprising distinct spikes or micropulses separated in time.

The output optical energy may be substantially zero during time intervals between spikes in the effective portion 120 of the output optical energy distribution. Further, a relatively small amount of output optical energy lies below the ablation threshold 125, and essentially no tail portion of the output optical energy distribution is present. For practical purposes, substantially all of the output optical energy distribution is effective for cutting tissue. The optical resonator of the present invention can be considered, by some accounts and in some instances, to produce virtually no wasted additional output optical energy that would contribute to pain for a patient and to a reduction in overall energy efficiency of the optical resonator. The optical resonator of the present invention can be considered, in other instances, to produce a reduced amount, relative to the discussed prior-art system, of wasted additional output optical energy that would contribute to pain for a patient and to a reduction in overall energy efficiency of the optical resonator. As such, the invention can provides for more effective (not just more efficient) cutting, so that a given volume of material can be removed with less energy and with concomitant increased comfort to the patient.

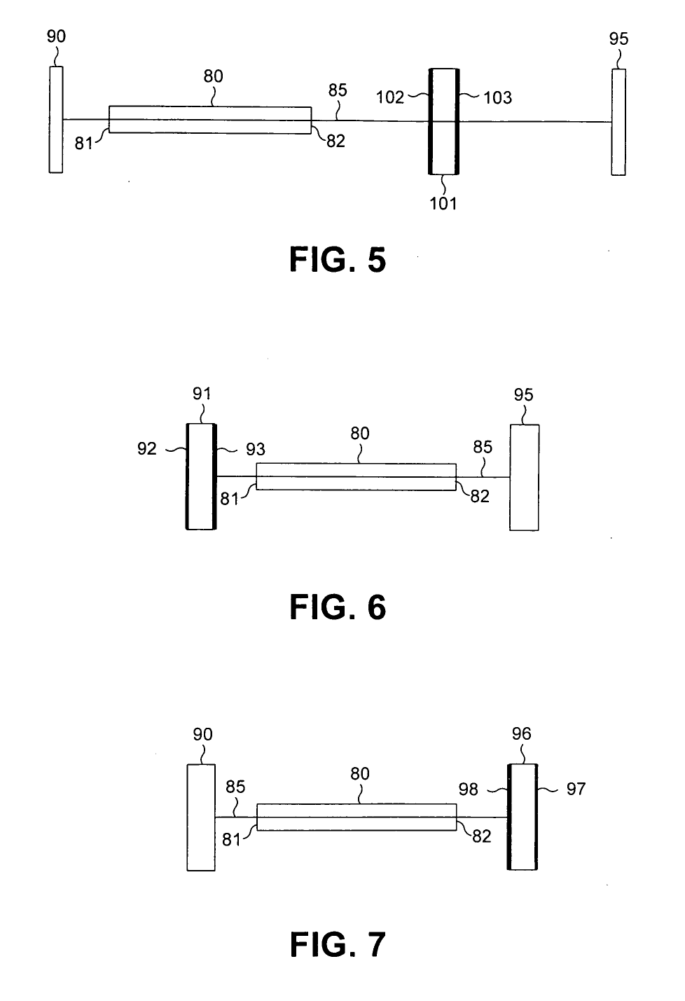

The optical resonator of the present invention may be implemented in other forms different from the embodiment of FIG. 3. For example, FIG. 5 illustrates an embodiment of the present invention comprising, as before, a laser rod 80 having an optical axis 85, an HR 90, and an OC 95. The laser rod 80 has respective first and second ends 81 and 82. A modulator 101 is disposed in a vertical orientation in the path of the optical axis between the second end 82 of the laser rod 80 and the OC 95. More precisely, the modulator 101, which may be fabricated of Fe:ZnSe as already described, may have first and second parallel surfaces that are normal to the optical axis, the first and second surfaces being coated with respective first and second anti-reflective coatings 102 and 103. (That is, normal to the first and second parallel surfaces is parallel with the optical axis.) In a modified embodiment (not shown), the modulator 101 may be disposed in the path of the optical axis between the HR 90 and the first end 81 of the laser rod 80.

In another embodiment of the present invention, the modulator may be incorporated into the HR or the OC. FIG. 6 illustrates the former case, depicting again the laser rod 80 with respective first and second ends 81 and 82, the optical axis 85, and the OC 95. The HR 91 in FIG. 6 is modified to be formed of Fe:ZnSe in order to provide a function similar to that provided by the modulator 101 in FIG. 5 and the modulator 100 in FIG. 3. The HR 91, which may be formed of zinc selenide material known to be transparent at a wavelength of 3 microns, may be doped with iron to form the modulator. The HR 91 in the illustrated embodiment is coated with an anti-reflective coating 93 on a side facing the laser rod 80 and is mirrored (shown with reference designator 92) on an opposite side.

In yet another embodiment of the present invention, the modulator may be incorporated into the OC. FIG. 7 illustrates such an embodiment, depicting again the laser rod 80 with respective first and second ends 81 and 82, the optical axis 85, and the HR 90. As with the HR 91 in FIG. 6, the OC 96 in FIG. 7 is modified to be formed of Fe:ZnSe in order to provide a function similar to that provided by the modulator 101 in FIG. 5 and the modulator 100 in FIG. 3. When the OC 96 is formed of zinc selenide material, it may be doped with iron to form the modulator. The OC 96 in the illustrated embodiment is coated with an anti-reflective coating 98 on a side facing the laser rod 80 and is mirrored (shown with reference designator 97) on an opposite side.

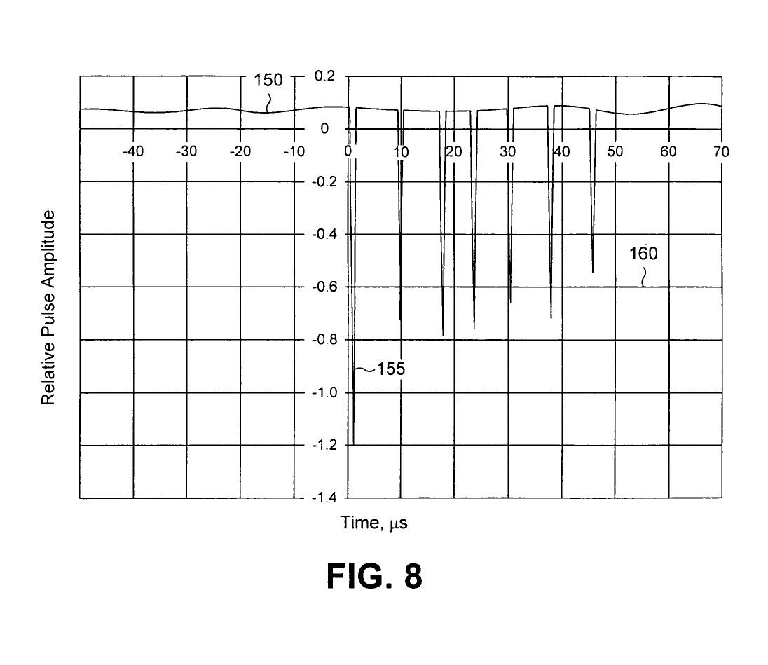

Continuing with the description above relative to FIG. 4, FIG. 8 illustrates an oscilloscope trace of an output optical energy waveform generated by an embodiment of the present invention. The oscilloscope trace is similar to the chart of FIG. 4, but is presented in an expanded time scale. The waveform, which has a negative polarity in the present example, has a quiescent value 150 of nearly zero, less than 0.1 in relative amplitude in this example. An active portion of the output optical energy waveform commences with an initial micropulse 155 having relative amplitude of approximately −1.2. Subsequent pulses, occurring at intervals of roughly 10 microseconds (μs), have relative amplitudes ranging from about −0.75 to −0.55, and all but one of these subsequent pulses have amplitudes more negative than −0.6, which may represent an ablation threshold 160 in this instance. It should be noted that the waveform possesses essentially no wasted tail energy and that a significant essentially-zero energy time interval exists between each of the energy pulses.

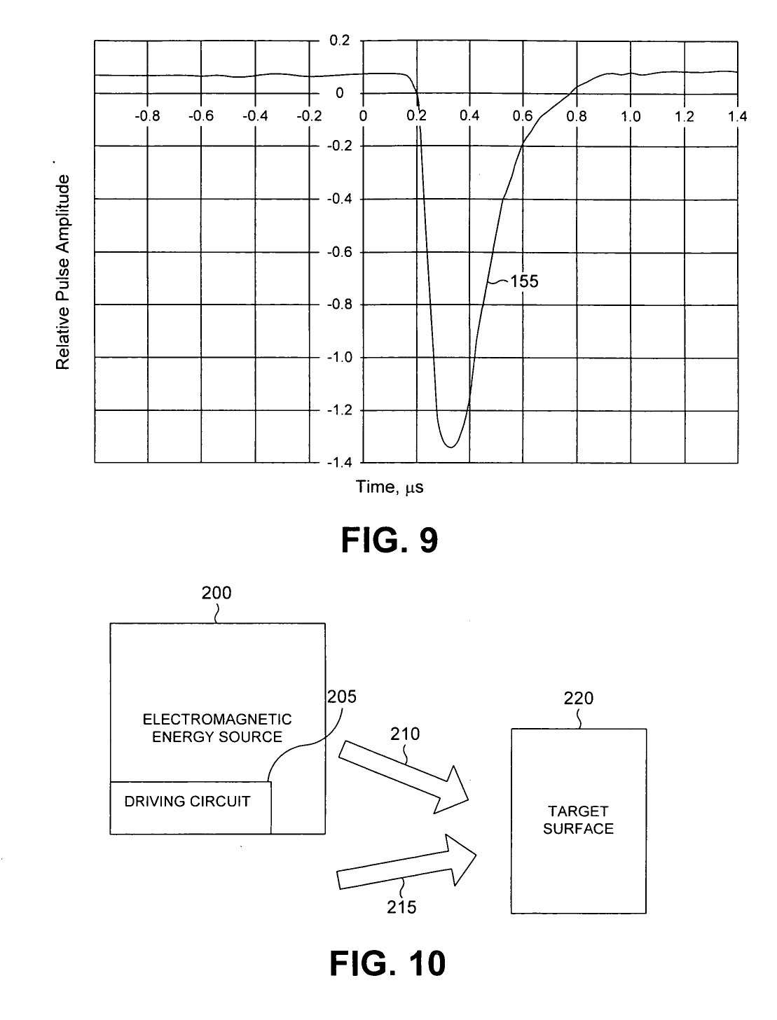

FIG. 9 is another oscilloscope trace of an energy waveform similar to that of FIG. 8, but on a still more expanded time scale than that of FIG. 4 or FIG. 8, produced by an embodiment of the present invention. The illustrated trace, which may correspond approximately to the initial micropulse 155 of FIG. 8, is shown to rise steeply from a small (less than 0.1 unit of relative amplitude) quiescent level to a relatively large (negative) amplitude of about −1.3 units and to decay quickly to the quiescent level. The pulse is observed to be isolated in time from any other pulses.

Additional embodiments of the present invention may be incorporated into a variety of laser systems. Systems of the present invention may comprise, for example, either an Er, Cr:YSGG solid state laser, which generates electromagnetic energy having a wavelength in a range of 2.70 to 2.80 microns, or an erbium, yttrium, aluminum garnet (Er:YAG) solid state laser, which generates electromagnetic energy having a wavelength of 2.94 microns. The Er, Cr:YSGG solid state laser may generate optical energy with a wavelength of approximately 2.78 microns, and optical energy produced by the Er:YAG solid state laser may have a wavelength of approximately 2.94 microns. According to one alternative embodiment, the laser rod 80 in FIG. 3, for example, may comprise a YAG crystal, and impurities in the crystal may comprise erbium impurities. A variety of other possibilities exists, a few of which are set forth in a book, Solid-State Laser Engineering, Fourth Extensively Revised and Updated Edition, by Walter Koechner, published in 1996, the contents of which are expressly incorporated herein by reference.

Other possible laser systems include an erbium, yttrium, scandium, gallium garnet (Er:YSGG) solid state laser, which generates electromagnetic energy having a wavelength in a range of 2.70 to 2.80 microns; an erbium, yttrium, aluminum garnet (Er:YAG) solid state laser, which generates electromagnetic energy having a wavelength of 2.94 microns; chromium, thulium, erbium, yttrium, aluminum garnet (CTE:YAG) solid state laser, which generates electromagnetic energy having a wavelength of 2.69 microns; erbium, yttrium orthoaluminate (Er:YAL03) solid state laser, which generates electromagnetic energy having a wavelength in a range of 2.71 to 2.86 microns; holmium, yttrium, aluminum garnet (Ho:YAG) solid state laser, which generates electromagnetic energy having a wavelength of 2.10 microns; quadrupled neodymium, yttrium, aluminum garnet (quadrupled Nd:YAG) solid state laser, which generates electromagnetic energy having a wavelength of 266 nanometers; argon fluoride (ArF) excimer laser, which generates electromagnetic energy having a wavelength of 193 nanometers; xenon chloride (XeCl) excimer laser, which generates electromagnetic energy having a wavelength of 308 nanometers; krypton fluoride (KrF) excimer laser, which generates electromagnetic energy having a wavelength of 248 nanometers; and carbon dioxide (C02), which generates electromagnetic energy having a wavelength in a range of 9 to 11 microns.

Certain energy (e.g., laser) emitting systems may employ fluid outputs in addition to electromagnetic (e.g., optical) energy to accomplish treatment (e.g., cutting) as is described, for example, in U.S. Pat. No. 6,288,499 entitled ELECTROMAGNETIC ENERGY DISTRIBUTIONS FOR ELECTROMAGNETICALLY INDUCED MECHANICAL CUTTING, the entire contents of which are incorporated herein by reference including all structures and methods, such as pulse particulars, for operation with the present invention.

FIG. 10 is a block diagram showing a fluid output used in combination with an electromagnetic energy source (e.g., a laser) having a driving circuit, which may comprise, for example, one or more diodes or flashlamps, in accordance with an implementation of the present invention. The output energy distributions of the present invention can be useful for maximizing a cutting effect of an electromagnetic energy source 200, such as a laser driven by a driving circuit 205, directed into a distribution (e.g., an atomized distribution) of fluid particles 215 above a target surface 220.

Such an apparatus for directing electromagnetic energy into an atomized distribution of fluid particles above a target surface is disclosed in the above-referenced U.S. Pat. No. 6,288,499 or U.S. Pat. No. 6,544,256, entitled ELECTROMAGNETICALLY INDUCED CUTTING WITH ATOMIZED FLUID PARTICLES FOR DERMATOLOGICAL APPLICATIONS, the entire contents of which are hereby incorporated by reference including all structures and methods, such as pulse particulars, for operation with the present invention. Referring again to FIG. 4, high-intensity leading micropulses, such as the large initial micropulse 115, and/or micropulses having characteristics of the type which follow, may impart relatively large amounts of energy into the fluid particles, which may comprise, for example, water, to thereby expand the fluid particles and apply disruptive (e.g., mechanical) cutting forces to a target surface 220 (FIG. 10). The effective portion 120 (FIG. 4) of micropulses, e.g., following the optional large initial micropulse 115, have been found to enhance cutting efficiency. According to the present invention, a single large leading micropulse, e.g., the large initial micropulse 115, may be generated. Alternatively, two, three, four, or more large leading micropulses may be generated. As another alternative, a magnitude of the large initial micropulse 115 or pulses may be reduced any amount down to a magnitude of the following micropulses, or omitted.

In view of the foregoing, it will be understood by those skilled in the art that the present invention can facilitate cutting, ablating, impartation of disruptive forces onto, or treatment of tissue in medical/dental applications with an increase in patient comfort when compared with cutting using conventional devices. The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. An optical resonator, comprising:
a laser rod having an optical axis;
a high-reflectivity optical element disposed within a path of the optical axis near a first end of the laser rod;
an output coupling element disposed within the path of the optical axis nearer to a second end of the laser rod than to the first end of the laser rod; and
a modulator disposed in the path of the optical axis, the modulator being adapted to influence energy pulses emitted by the optical resonator, whereby substantially all energy pulses having an amplitude not exceeding an ablation threshold have an amplitude significantly less than the ablation threshold.

2. The optical resonator as set forth in claim 1, wherein the modulator comprises iron-doped zinc selenide (Fe:ZnSe).

3. The optical resonator as set forth in claim 2, wherein the modulator is disposed between the second end of the laser rod and the output coupling element.

4. The optical resonator as set forth in claim 3, wherein the modulator is disposed at a Brewster angle to the optical axis.

5. The optical resonator as set forth in claim 3, wherein the modulator is disposed in a vertical orientation relative to the optical axis.

6. The optical resonator as set forth in claim 5, wherein the modulator comprises at least one anti-reflective coating.

7. The optical resonator as set forth in claim 2, wherein the modulator is disposed between the high-reflectivity optical element and the first end of the laser rod.

8. The optical resonator as set forth in claim 7, wherein the modulator is disposed at a Brewster angle relative to the optical axis.

9. The optical resonator as set forth in claim 7, wherein the modulator is disposed in a vertical orientation relative to a horizontally-oriented optical axis.

10. The optical resonator as set forth in claim 9, wherein:
the modulator comprises first and second parallel surfaces having normals parallel to the optical axis; and
the first and second surfaces are coated with anti-reflective coatings.

11. The optical resonator as set forth in claim 1, wherein the modulator comprises a portion of the high-reflectivity optical element.

12. The optical resonator as set forth in claim 1, wherein the modulator comprises a portion of the output coupling element.

13. The optical resonator as set forth in claim 1, wherein the laser rod comprises an erbium, chromium, yttrium, scandium, gallium, garnet crystal.

14. A medical laser capable of generating optical energy, the medical laser comprising:
a laser rod having an optical axis;
a high-reflectivity optical element disposed within a path of the optical axis near a first end of the laser rod;
an output coupling element disposed within the path of the optical axis nearer to a second end of the laser rod than to the first end of the laser rod; and
a saturable absorber disposed in the path of the optical axis, wherein a majority of the optical energy generated by the medical laser has a level greater than an ablation threshold of tissue.

15. The medical laser as set forth in claim 14, wherein the saturable absorber comprises crystalline iron-doped zinc selenide.

16. The medical laser as set forth in claim 14, wherein:
the saturable absorber comprises first and second parallel surfaces having normals oriented at a Brewster angle with the optical axis; and
the saturable absorber is disposed between the second end of the laser rod and the output coupling element.

17. The medical laser as set forth in claim 14, wherein:
the saturable absorber comprises first and second parallel surfaces having normals oriented at a Brewster angle with the optical axis; and
the saturable absorber is disposed between the first end of the laser rod and the high-reflectivity optical element.

18. The medical laser as set forth in claim 14, wherein:
the saturable absorber comprises first and second parallel surfaces having normals oriented parallel with the optical axis;
the saturable absorber is disposed between the second end of the laser rod and the output coupling element; and
the saturable absorber is coated with anti-reflective coatings on the first and second parallel surfaces.

19. The medical laser as set forth in claim 14, wherein the saturable absorber comprises a portion of the high-reflectivity optical element.

20. The medical laser as set forth in claim 14, wherein the saturable absorber comprises a portion of the output coupling element.

21. The medical laser as set forth in claim 14, wherein the laser generates electromagnetic energy comprising one of a wavelength within a range from about 2.69 to about 2.80 microns and a wavelength of about 2.94 microns.

22. The medical laser as set forth in claim 14, wherein the laser comprises one of an Er:YAG, an Er:YSGG, an Er, Cr:YSGG and a CTE:YAG laser.

23. The medical laser as set forth in claim 14, further comprising a fluid output configured to place fluid particles into a volume in close proximity to a target surface.

24. The apparatus as set forth in claim 23, wherein:
the fluid output comprises an atomizer configured to place atomized fluid particles into a volume above the target surface; and
the laser is configured to impart relatively large amounts of energy into the atomized fluid particles in the volume above the target surface to thereby expand the atomized fluid particles and impart disruptive forces onto the target surface.

25. The apparatus as set forth in claim 24, wherein the target surface comprises one of tooth, bone, cartilage and soft tissue.

26. The apparatus as set forth in claim 25, wherein the fluid particles comprise water.

27. The apparatus as set forth in claim 14, wherein the ablation threshold is an ablation threshold of hard tissue.

28. The apparatus as set forth in claim 27, wherein the ablation threshold is an ablation threshold of tooth tissue.

29. The apparatus as set forth in claim 14, wherein the ablation threshold is an ablation threshold of soft tissue.
